(19) United States
(12) Patent Application Publication
(10) Pub. No.: US 2022/0031492 A1
(43) Pub. Date: Feb. 3, 2022

(54) DEVICE AND METHOD FOR CORRECTING POSTURE

(71) Applicants: Sergio Peñaloza, Santa Monica, CA (US); Cameron Aston, Malibu, CA (US)

(72) Inventors: Sergio Peñaloza, Santa Monica, CA (US); Cameron Aston, Malibu, CA (US)

(21) Appl. No.: 16/983,416

(22) Filed: Aug. 3, 2020

(51) Int. Cl. A61F 5/02

(52) U.S. Cl. CPC A61F 5/026

(57) ABSTRACT

A device to promote proper spinal alignment during an exercise having a lightweight, rigid bar inserted into a sheath formed on the outside of a support, such as a vest, which secures the bar to the user. The bar extends from the top of the support, reaching from the bottom of the spine up to the back of the user's head. The vest allows for the bar to be held against the user's body while leaving the head free to move. The device can be worn while stationary or moving, including during physical activities, and more particularly during exercises. The device provides tactile information of proper spinal alignment during an activity, so that alignment can be corrected and proper alignment can be learned in order to reduce the risk of injury.

### TECHNICAL FIELD

The present invention is in the technical field of apparel, in particular, the present invention is in the technical field of vest type apparel. More particularly, the invention relates to vest type apparel for back support.

### BACKGROUND

When performing an exercise, technique is just as important, if not more important, than the number of repetitions or sets, or the amount of resistance experienced during the exercise. Technique can refer to the exerciser's posture, the motion or mechanics of the body movement, or both. In fact, improper technique, and specifically improper posture, can lead to injury. Yet, many exercisers have improper technique without even knowing it.

In order to know whether an exerciser's technique is proper or not, the exerciser would have to hire a personal trainer and the have the personal trainer instruct the exerciser regarding the proper technique. Hiring a personal trainer can be expensive and decreases the flexibility in time for when the exerciser can perform his or her exercises. Furthermore, a mirror may be required for the exerciser to check to make sure his or her technique is correct.

For the foregoing reasons there is a need for a device that can help exercises maintain proper posture while performing an exercise without the need of a personal trainer or mirrors.

### SUMMARY

The present invention is a postural corrective device to promote proper spinal alignment for users to wear while sitting, standing, or doing physical activity, in particular, exercising. The device comprises a lightweight and rigid bar that provides tactile information to indicate proper, straight spinal alignment, which allows users to realize where posture corrections need to be made before performing an exercise; and a support to hold the bar against the user. Preferably, the support is a garment, such as a vest or jacket with a retaining sleeve along the back side. The bar is adjustable and removable by sliding in and out of a retaining sleeve. More particularly, the device provides a single attachment method along the torso so that the bar fits snugly and comfortably against the user's back. This ensures that the bar can follow the user's movements during activities such as exercising, including tilting from side to side and rotating, and therefore the device remains in the correct position with the top of the bar behind the user's head. Furthermore, the garment has a single attachment element, such as a zipper, along the vest, allowing for easy closure and centered alignment of the device. In addition, a vest being secured over the user's shoulders prevents the device from slipping downward during activities such as jumping or running. This invention is simpler than prior art devices, in that there is a single rigid bar that is inserted into a single fabric retaining sleeve, whereby the height of the bar can be adjusted by sliding into and out of the retaining sleeve, and a series of strap attachments are not needed. Furthermore, the rigid bar is not sewn into the sleeve, and is not connected to any strap attachments, and is therefore adjustable and removable during all activities including laying down.

An object of the present invention is to provide a postural corrective device that can be worn during physical activities such as exercise or therapy.

Another object of the present invention is to provide a device designed to be worn during physical activity, to provide real-time awareness of posture during physical activity.

Another object of the present invention is to provide a device that works to keep the head upright, and be able to be used during exercise. It is adjustable, and comes in a range of sizes. The vest has a closure to ensure comfortable and proper fitting during movement.

### BRIEF DESCRIPTION OF DRAWINGS

FIG. 1 shows a perspective view of an embodiment of the present invention;

FIG. 2 shows a side elevation view of an embodiment of the present invention;

FIG. 3 shows a rear elevation view of an embodiment of the present invention;

FIG. 4 shows an exploded view of an embodiment of the present invention;

FIG. 5 shows a perspective view of an embodiment of the bar.

### DETAILED DESCRIPTION OF THE INVENTION

The detailed description set forth below in connection with the appended drawings is intended as a description of presently-preferred embodiments of the invention and is not intended to represent the only forms in which the present invention may be constructed or utilized. The description sets forth the functions and the sequence of steps for constructing and operating the invention in connection with the illustrated embodiments. It is to be understood, however, that the same or equivalent functions and sequences may be accomplished by different embodiments that are also intended to be encompassed within the spirit and scope of the invention.

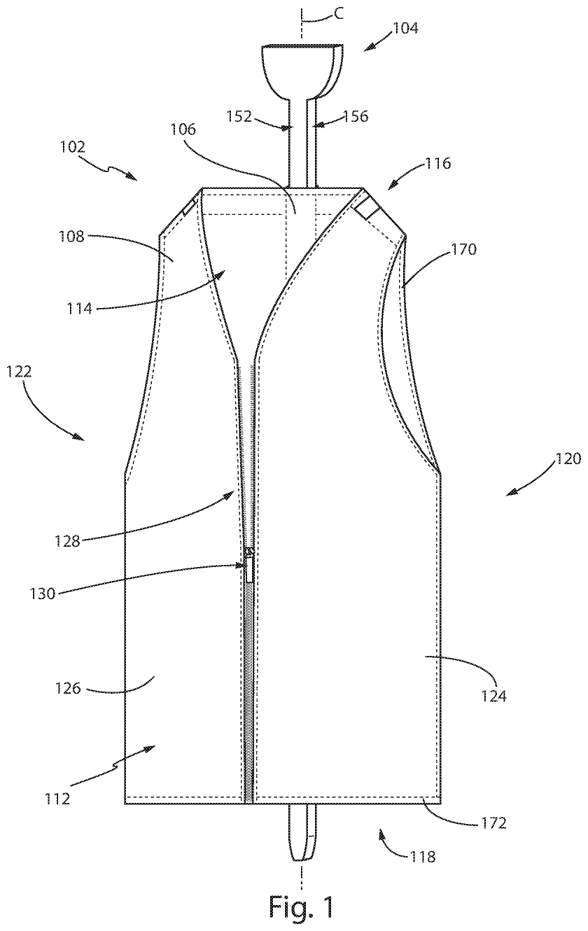

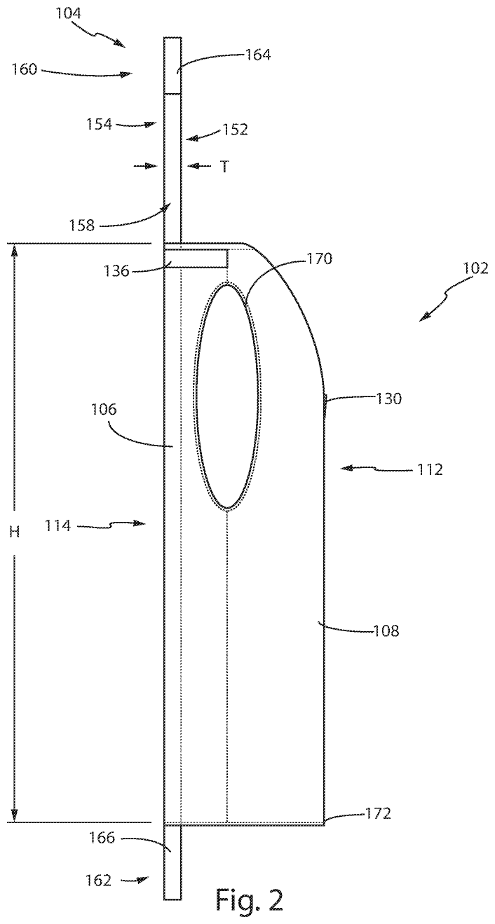

The invention of the present application is directed towards a device 100 for correcting posture of a user during exercise to establish a proper posture for the exercise being performed. Preferably, the proper posture is maintained during at least one repetition of the exercise movement being performed. With reference to FIGS. 1 and 2, the device 100 comprises a support 102, and a bar 104 held against the user's body with the support 102. The support 102 is configured to be worn by the user and hold the bar 104 against the user's spine even during movement. As such, the support 102 comprises a holder 106 to hold the bar 104, and a body 108 to be donned by the user. The body 108 can be configured to be donned by the user by being secured around the user's torso. For example, the body 108 can be secured around the user's lumbar region, thoracic region, cervical region, over the shoulders, or any combination thereof.

The body 108 of the support 102 is defined by a front side 112, a back side 114 opposite the front side 112, a top 116 side adjacent to the front side 112 and the back side 114, and a bottom side 118 opposite the top side 116 and adjacent to the front side 112 and the back side 114, the body 108 defining a centerline C dividing the front side 112 and back side 114 equally into a left side 120 and a right side 122, wherein the left side and right side are substantially equivalent to each other, thereby essentially forming a mirror image of each other.

In the preferred embodiment, the front side 112 comprises a left body panel 124 and a right body panel 126 separated by an opening 128 that can be closed by a fastener 130. The opening 128 can be a partial opening in which a portion of the left body panel 124 and right body panel 126 are integrally formed on the front side 112, particularly towards the bottom side 118. Preferably, the opening 128 is a full opening in which the left body panel 124 and the right body panel 126 can be separated completely on the front side 112. The fastener 130 can be any type of closure used with garments, such as zippers, hook-and-loop fasteners, buttons, magnets, clips, hooks, and the like. Preferably, the support 102 is a garment that can be worn by the user, such as belt, weight belt, a strap, a harness, a shirt, a vest, a pullover, a jacket, and the like.

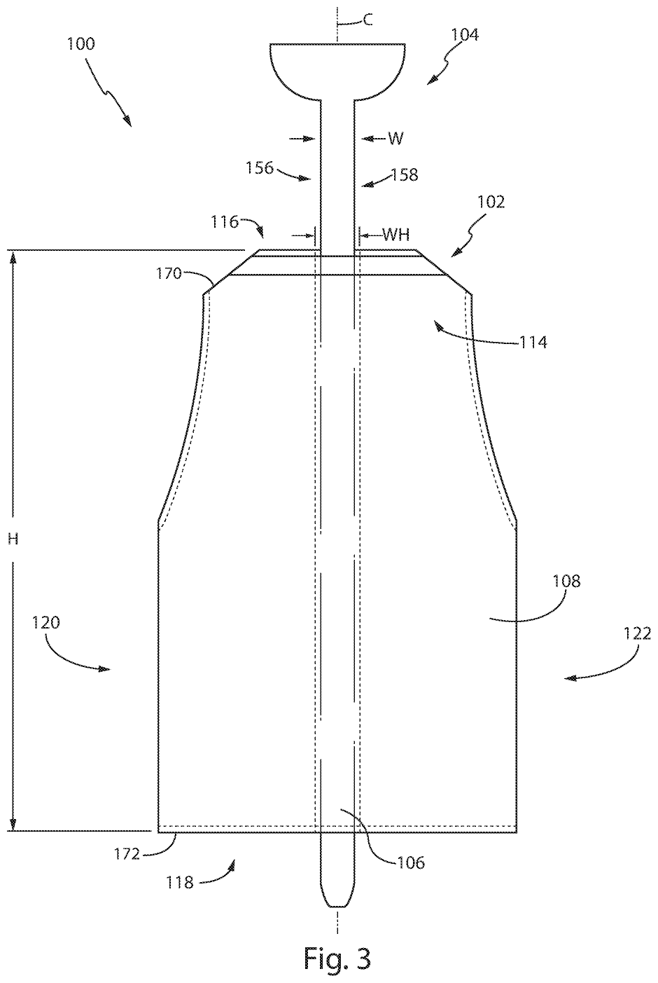

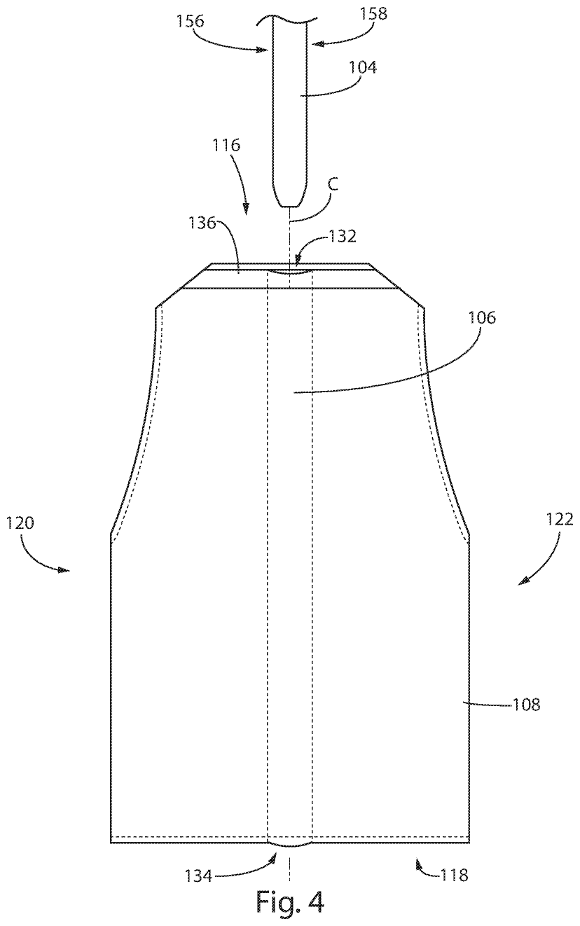

With reference to FIGS. 3 and 4, on the back side 114 of the body 108 is the holder 106. Preferably, the holder 106 is positioned on the back side 114 along the centerline C and configured to hold the bar 104 securely along the spine of the user. As such, the holder 106 can be a clasp, rings, straps, magnets, a sheath, and the like. Critically, the holder 106 is configured to hold the bar 104 at various positions along the body 108. In other words, the bar 104 can be held in place at various heights relative to the user. In addition, the holder 106 is configured to keep the bar 104 pressed against to the user along the thoracic region.

In the preferred embodiment, the support 102 is a garment worn by the user. More preferably, the support 102 is a top garment, such as a vest, shirt, or jacket. As such, the top garment generally covers the upper torso of the user from about the neckline to about the waist, including the trapezius, back, chest, abdominal regions, and the like. As a top garment, the support 102 is more aesthetically pleasing than simple straps and bands because the top garment looks like typical clothing rather than some kind of exercise device that would only be worn in the gym or the privacy of one's home. The top garment may have short sleeves as in a t-shirt, long sleeves as in a jacket, or be sleeveless as in a vest.

The holder 106 can be formed onto the body 108. For example, the body 108 can define a top opening 132 along the top side 116 through which the bar 104 can be inserted to define the holder 106. The material defining the top opening 132 keeps the bar 104 against the top side 116. More preferably, the body 108 also defines a bottom opening 134 along the bottom side 118 through which the bar 104 can exit the holder 106 once inserted through the top opening 132. The material defining the bottom opening 134 keeps the bar against the bottom side 118. Preferably, the support 102 is made of a flexible, breathable, elastic, and washable material, with a fastener 130 on the front side to ensure that the support 102 is secure and comfortable. The device 10 is an indicator when the user's posture is not properly aligned. As such, the material can be thin material sufficient to keep the bar 104 adjacent to the user's back. Using a top garment as the support 102 has the benefit of not having to significantly change manufacturing processes of clothing as the holder 106 can be added to top garments during the manufacturing process of typical top garments (albeit the material may be different). Therefore, the manufacturing process is simplified as typical top garment manufacturing procedures can be utilized and modified to add the holder 106.

In the preferred embodiment, the holder 106 extends substantially from the top side 116 of the body 108 to the bottom side 118 of the body 108 forming a sheath to cover a substantial portion of the bar 104. As such, in the preferred embodiment, the holder 106 provides a single holder for holding the bar 104 properly in place rather than multiple holding points. The sheath can have a height H ranging from about 20 inches to about 40 inches, and anywhere therebetween in approximately half inch increments. This range covers the most common shirt, jacket, vest lengths from youth to women to men to unusually tall men. Preferably, the height H can be about 23 inches to about 36 inches. More preferably, the height H can be about 25 inches to about 33 inches. Due to the height H of the holder 106 relative to the length of the body 108, the bar 104 remains in contact with the user's back even during movement.

The material of the sheath causes the bar 104 to compress against the user's spine. As such, in the preferred embodiment, the holder 106 is a sheath defining a top opening 132 along the top side 116, and a bottom opening 134 along the bottom side 118. In the preferred embodiment, the holder 106 can have bartack stitches to reinforce the holder 106 on the body 108 of the support 102. The material at the top opening 132 can be folded back on to the holder 106 to ensure smooth insertion of the bar 104. The remaining portion of the holder 106 can be stitched to the body 108 with flatlock stitching.

In some embodiments, a reinforcement strap 136 is positioned on the back side 114 of the body 108 along the top side 116, and across the holder 106 adjacent to the top opening 132. The reinforcement strap 136 applies additional pressure against the bar 104 to facilitate holding the bar 104 in place.

As such, in the preferred embodiment, the amount and extent of resistive or compressive force applied on the bar 104 from the holder 106 and the reinforcement strap 136 is sufficient to keep the bar 104 in place against the user's back during normal use, while still allowing the user to move the bar 104 in and out of the holder 106 (up and down). With the holder 106 extending the full length of the body 108 from the top side 116 to the bottom side 118, the surface area making contact with the bar 104 is maximized, which in turn maximizes the resistive force to prevent the bar from slipping out of holder 106. The reinforcement strap 136 applies a second form of compression against the bar 104. Finally, the body 108 itself can apply a third from of compression or resistance against the bar 104. For example, in the open configuration, the body 108 can be loose against the user. When the user fastens the opening 128 with the fastener 130, the body 108 of the support 102 compresses around the user's upper torso. This compression also causes compression against the bar 102 making it very difficult for the bar 102 to move in or out (up or down) along the holder 106. Therefore, when the support is properly donned by the user, the bar 104 experiences the most amount of compression making it unlikely to move within the holder 106.

In some embodiments, the holder 106 need not extend continuously, the full length of the body 108. For example, the holder 106 can be a strap along the top side 116 and a strap along the bottom side 118. In some embodiments, there may be a series of straps intermittently spaced apart along the length of the body 108.

Preferably, the body 108 and the holder 106 are made from elastic material to facilitate the compression of the bar against a spine of the user while maintaining comfort and mobility for the user. Due to the elasticity of the body 108 and the holder 106, the user is allowed to move (i.e. bend, twist, expand, and the like) with minimal discomfort. The material composition and weight provide the desired combination of durability, flexibility, texture or feel, and temperature control for exercise.

For example, the body 108 and the holder 106 may comprise polyester, nylon, cotton, spandex (elastane), and the like, or any combination thereof. In some embodiments, the body 108 and the holder 106 can comprise about 60 percent to about 90 percent polyester and about 10 percent to about 40 percent spandex (elastane), such as Lycra®. Preferably, the body 108 and the holder 106 comprise about 70 percent to about 85 percent polyester and about 15 percent to about 30 percent spandex (elastane). More preferably, the body 108 and the holder 106 comprise about 75 percent to about 80 percent polyester and about 20 percent to about 25 percent spandex (elastane). The weight of the material used to make the body 108 and holder 106 can be about 250 GSM to about 350 GSM. Preferably, the weight of the material is about 275 GSM to about 325 GSM. More preferably, the weight of the material is about 290 GSM to about 310 GSM. For example, in one preferred embodiment, the body 108 and holder 106 can comprise about 78 percent polyester and about 22 percent of LYCRA® spandex material with a weight of about 300 GSM.

Although the vest is the preferred embodiment for the support 102, the holder 106 can be held to the body by other types of supports 102. For example, removing the panels of the vest, the holder 106 can be held in place simply as a shoulder harness 170 with a waist strap 172. As such, the support 102 can be in the form of a chest harness, a back pack, a back brace, and the like.

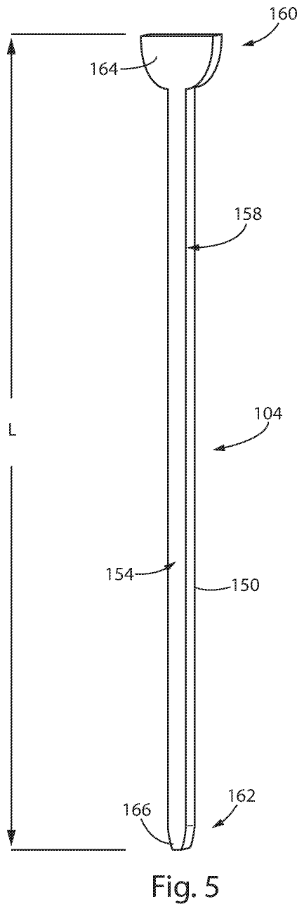

With reference to FIG. 5, the bar 104 comprises a shaft 150 having an anterior surface 152, a posterior surface 154 opposite the anterior surface 152, a first side 156 adjacent to the anterior surface 152 and the posterior surface 154, and a second side 158 opposite the first side 156 and adjacent to the anterior surface 152 and the posterior surface 154, a superior side 160 adjacent to the anterior surface 152, the posterior surface 154, the first side 156, and the second side 158, and an inferior side 162 opposite the superior side 160, and adjacent to the anterior surface 152, the posterior surface 154, the first side 156, and the second side 158, wherein the bar 104 defines a length L measured from the superior side 160 to the inferior side 162, a width W measured from the first side 156 to the second side 158, and a thickness T defined from the anterior surface 152 to the posterior surface 154. Preferably, the anterior surface 152 and the posterior surface 154 are flat surfaces, thereby defining a planar shaft 150. More preferably, the thickness T of the bar 104 is substantially uniform throughout the bar 104 so that the anterior surface 152 and the posterior surface 154 do not merge together to form a sharp edge. A flat anterior surface 152 is comfortable and conducive for maintaining its position against the user's spine. Nonetheless, other shapes can be used for the anterior surface 152 and the posterior surface 154. For example, in some embodiments, the anterior surface can be concave to accommodate the spine by creating a small channel. In some embodiments, the posterior surface can be convex or rounded.

The superior side 160 terminates at a head 164. Preferably, the head 164 is a flanged head 164 that provides tactile information of proper placement or positioning of the user's head for proper posture. The bar 104 is not attached to the user's head, so that the head is free to move and rotate. The flanged head may be T-shaped, Y-shaped, U-shaped, semi-circular, circular, square, rectangular, and the like, such that the width W of the bar 104 at the flanged head 164 is greater than the width W of the bar 104 at the shaft 150. In some embodiments, the posterior surface 154 can be concave to match the contour of the back of the head of the user.

The inferior side 162 of the bar 104 terminates at a tail 166. Preferably, the tail 166 is a tapered tail. As such, the width W of the bar 104 at the tapered tail 166 is less than the width W of the bar at the shaft 150.

The length L of the bar 104 is greater than the length of the support 102. Specifically, the length L of the bar 104 is greater than a distance D from the top opening 132 to the bottom opening 134. As such, when the bar 104 is inserted into the holder 106, the head 164 of the bar 104 extends above the top side 116 of the body 108, while the tail 166 of the bar 104 extends below the bottom side 118 of the body 108. Therefore, the length of the holder measured from the top side 116 to the bottom side 118 is less than the length L of the bar 104, and in particular, the length of the shaft 150.

The width WH of the holder 106 as measured from the left side 120 to the right side 122 is substantially the same as the width W of the bar 104 at the shaft 150. Due to the elasticity of the holder, the width WH of the holder 106 can be slightly larger or slightly smaller than the width W of the shaft 150. Combined with the disclosed height H of the holder 106, additional points of securement of the holder 106 to the support 102 is not required.

By way of example only, the length L of the bar 104 can range from about 25 inches to about 45 inches. For particularly tall people, the bar 104 can be made taller. Preferably, the length L of the bar 102 is about 28 inches to about 40 inches. More preferably, the length L of the bar 104 is about 30 inches to about 35 inches. The width W of the bar 104 can range from about 0.5 inch to about 5 inches. Preferably, the width W of the bar 104 is about 1 inch to about 4 inches. More preferably, the width W of the bar 104 is about 2 inches to about 3 inches. The thickness T of the bar 104 depends on the material used. The bar 104 has to be able to maintain sufficient stiffness such that if the user bends his or her upper torso, the bar 104 does not bend with the user. The purpose of the bar is to maintain its straightness as a gauge to determine whether the user is maintaining proper posture. With these guidelines in mind, the thickness T of the bar 104 can range from about one-eighth of an inch to about one inch. Preferably, the thickness T of the bar 104 is about one-quarter of an inch to about three-quarters of an inch. More preferably, the thickness T of the bar is about half an inch. In the preferred embodiment, the thickness T of the bar 102 is consistent throughout the length L of the bar 104. In addition, in the preferred embodiment, the bar 102 is straight when viewed from the side as shown in FIG. 2, meaning the anterior surface 152 and the posterior surface 154 remain in their original planes throughout the entire length L of the bar 102.

Preferably, the bar 104 comprises a lightweight and rigid composite plastic. The bar 104 can be made of other rigid material such as plastic, wood, metal, carbon fiber, fiberglass, and the like, or any combination thereof. The bar 104 is sufficiently stiff so as to resist bending when the user bends his or her body while wearing the device 100 properly.

In use, the user can slide the bar 104 into the holder 106 of the support 102 through the top opening 132 until the tail 166 emerges from the bottom opening 134. The user can don the support 102 and leave the support 102 opened or partially opened. The bar 104 can be adjusted so that the head 164 of the bar is adjacent to the head of the user, approximately near the occipital region. At this position, the inferior side 162 of the bar 104 should be adjacent to the coccyx of the user. The support 102 can be fully fastened to secure the bar 102 in position. Once fully fastened, the bar 104 is aligned longitudinally with the user's spine. The bar 104 can also be adjusted in place while the support is fully fastened. To attain proper posture, the user stands up straight and adjusts his or her posture until his or her coccygeal region touches a portion of the inferior side 162. The user also brings his or head backward until the back of the user's head touches the head 164 of the bar 104. In this position, the user is considered to have proper posture as used in this application. As the user performs an exercise in which proper technique requires proper posture, the user can pay attention to whether his or her head is maintaining contact with the head 164 of the bar 104, and whether his or her coccygeal region is maintaining contact with the inferior side 162 of the bar. If the user's head is not touching the head 164 of the bar 104, then the user knows that his or her head is too far forward and needs to bring his or her head backward. If the coccygeal region is not touching the inferior side 162 of the bar 104, the back may be hunched forward, and the user will know to bring his or her shoulders back and stand up taller until the coccygeal region touches the inferior side 162 of the bar 102. Therefore, the user is able to correct his or her posture before or during the exercise by adjusting his or her head, shoulder, and/or back before or during an exercise.

By way of example only, the types of exercises in which proper posture would be beneficial include, but are not limited to, squats, lunges, planks, push-ups, lifts, presses, cycling, and the like. Aside from actual exercise, the user can use the device 100 simply to correct posture while sitting, standing, or engaging in any other type of physical activity. As such, the device can be used at home, at the gym, during physical therapy, while playing sports, and the like.

The foregoing description of the preferred embodiment of the invention has been presented for the purposes of illustration and description. It is not intended to be exhaustive or to limit the invention to the precise form disclosed. Many modifications and variations are possible in light of the above teaching. It is intended that the scope of the invention not be limited by this detailed description, but by the claims and the equivalents to the claims appended hereto.

1. A device for correcting a posture of a user during exercise, the device comprising:
a) a support, wherein the support comprises
i) a body, defined by a front side, a back side opposite the front side, a top side adjacent to the front side and the back side, and a bottom side opposite the top side and adjacent to the front side and the back side, the body defining a sagittal plane dividing the front side and back side equally into a left side and a right side, the front side comprising a fastener, a left body panel, and a right body panel, wherein the left body panel and the right body panel are configured to cover an upper torso of a user from a neckline of a user to a waistline of the user,
ii) a sheath, positioned on the back side along the sagittal plane extending from the top side to the bottom side, wherein the sheath defines a top opening along the top side, and a bottom opening along the bottom side, the sheath having a height of about 25 inches to about 33 inches, and
iii) a reinforcement strap across the sheath at the top side; and
b) a bar, comprising:
i) a planar shaft having a flat anterior surface, a flat posterior surface opposite the flat anterior surface, a first side adjacent to the flat anterior surface and the flat posterior surface, and a second side opposite the first side and adjacent to the flat anterior surface and the flat posterior surface, a superior side adjacent to the flat anterior surface, the flat posterior surface, the first side, and the second side, and an inferior side opposite the superior side, and adjacent to the flat anterior surface, the flat posterior surface, the first side, and the second side,
ii) a flanged head at the superior side of the planar shaft protruding abruptly from the first side and the second side, and
iii) a tapered tail at the inferior side of the planar shaft opposite the flanged head, wherein the bar defines a length measured from the flanged head to the tapered tail, a width measured from the first side to the second side, and a thickness defined from the flat anterior surface and the flat posterior surface;
c) wherein a height of the sheath measured from the top side to the bottom side is less than the length of the bar, and a width of the sheath is substantially the same as the width of the bar, and
d) wherein the body and the sheath are made from elastic material configured to compress the bar against a spine of the user.

2. A device for correcting a posture of a user during exercise, the device comprising:
a) a support, comprising a sheath wherein the sheath defines a top opening, and a bottom opening, the sheath having a height of about 20 inches to about 40 inches; and
b) a bar configured to fit through the top opening and the bottom opening, the bar comprising an anterior surface, a posterior surface opposite the anterior surface, a first side adjacent to the anterior surface and the posterior surface, and a second side opposite the first side and adjacent to the anterior surface and the posterior surface, a superior side adjacent to the anterior surface, the posterior surface, the first side, and the second side, and an inferior side opposite the superior side, and adjacent to the anterior surface, the posterior surface, the first side, and the second side, wherein the bar defines a length measured from the superior side to the inferior side, a width measured from the first side to the second side, and a thickness defined from the anterior surface to the posterior surface, wherein the length of the bar is greater than the height of the sheath, wherein the thickness of the bar is uniform throughout the bar, and wherein a flanged head abruptly projects laterally away from the first side and second side of the bar.

3. (canceled)

4. The device of claim 3, wherein the anterior surface is flat and the posterior surface is flat.

5. (canceled)

6. The device of claim 4, wherein the inferior side of the bar comprises a tapered tail.

7. (canceled)

8. The device of claim 2, wherein the sheath comprises elastic material to compress the bar.

9. The device of claim 8, wherein the support comprises a shoulder harness and a waist strap.

10. The device of claim 8, wherein the support is a top garment, comprising a left body panel, and a right body panel, wherein the left body panel and the right body panel are configured to cover an upper torso of a user from a neckline of the user to a waist of the user.

11. The device of claim 10, wherein the top garment is a vest, and the left body panel and the right body panel define an opening, wherein the vest further comprises a fastener configured to close the opening.

12. The device of claim 11, wherein the sheath is positioned on a back side of the vest along the centerline extending from a top side of the vest to a bottom side of the vest, wherein the top opening of the sheath is at the top side of the vest and the bottom opening of the sheath is at a bottom side of the vest.

13. The device of claim 12, further comprising a reinforcement strap across the sheath at the top side.

14. The device of claim 8, wherein the support is a vest comprising about 60 percent to about 90 percent polyester, and 10 percent to about 40 percent spandex, and has a weight of about 250 GSM to about 350 GSM.

15. The device of claim 8, wherein the support is a vest comprising about 75 percent to about 80 percent polyester, and 20 percent to about 25 percent spandex, and has a weight of about 290 GSM to about 310 GSM.

16. A method for correcting posture of a user during an exercise, comprising:
a) inserting a bar into a support, wherein the support defines a top opening, and a bottom opening, and the bar is configured to fit through the top opening and the bottom opening, wherein the bar defines a length measured from a superior side to an inferior side that is greater than a distance from the top opening and the bottom opening of the support, wherein the support comprises a left body panel, and a right body panel, wherein the left body panel and the right body panel are configured to cover an upper torso of a user from a neckline of a user to a waistline of the user;
b) donning the support such that the bar aligns with a spine of the user;
c) adjusting a posture of the user until a coccygeal region of the user touches the inferior side of the bar and a head of the user touches the superior side of the bar, whereby the posture of the user is corrected to establish a proper posture; and
d) performing an exercise.

17. The method of claim 16, further comprising maintaining the proper posture continuously during at least one repetition of the exercise.

18. The method of claim 16, wherein the superior side of the bar comprises a flanged head, and adjusting the head of the user to touch the superior side of the bar comprises touching the flanged head with the head of the user.

19. The method of claim 16, further comprising compressing the bar against a back of the user by closing a front side of the support.

20. The method of claim 19, wherein the support comprises about 60 percent to about 90 percent polyester, and 10 percent to about 40 percent spandex, and has a weight of about 250 GSM to about 350 GSM.
